(19) United States
(12) Patent Application Publication
(10) Pub. No.: US 2025/0327808 A1
(43) Pub. Date: Oct. 23, 2025

(54) IN-VITRO METHOD FOR SIMULATING AND ANALYZING BEHAVIOR OF AN OPHTHALMOLOGICAL DRUG IN AN EYE

(71) Applicant: LONZA LTD, Visp (CH)

(72) Inventors: Ahmad SEDIQ, Basel (CH); Wei Han TAN, Basel (CH); Christian WEINMANN, Basel (CH); Dhananjay JERE, Basel (CH); Hanns-Christian MAHLER, Loerrach (DE)

(21) Appl. No.: 18/837,324

(22) PCT Filed: Feb. 9, 2023
(86) PCT No.: PCT/EP2023/053247

§ 371 Date: Aug. 9, 2024

(30) Foreign Application Priority Data
Feb. 11, 2022 (EP) 22156329.9
Apr. 7, 2022 (EP) 22167213.2
Oct. 19, 2022 (EP) 22202597.5

(51) Int. Cl. G01N 33/58; G01N 1/40

(52) U.S. Cl. CPC G01N 33/582; G01N 1/4077; G01N 2001/4088; G01N 2500/20

(57) ABSTRACT

The present invention relates to a method and an apparatus for simulating and analyzing the behavior of a substance in an eye, in particular in the vitreous humor. Further, the present invention refers to a method of providing a vitreous humor, and to a buffer fluid for use in such a method and apparatus.

### CROSS-REFERENCE TO RELATED APPLICATIONS

This application is a U.S. national stage application of PCT/EP2023/053247 filed 9 Feb. 2023, which claims the benefit of, and relies on the filing date of, European Patent Application No. 22156329.9 filed 11 Feb. 2022, European Patent Application No. 22167213.2 filed 7 Apr. 2022, and European Patent Application No. 22202597.5 filed 19 Oct. 2022, the entire disclosures of which are incorporated herein by reference.

### TECHNICAL FIELD

The present invention relates to a method and an apparatus for simulating and analyzing the behavior of a substance in an eye, in particular in the vitreous humor. Further, the present invention refers to a method of providing a vitreous humor, and to a buffer fluid for use in such a method and apparatus.

### TECHNOLOGICAL BACKGROUND

Blinding diseases of the back of the eye such as age-related macular degeneration or diabetic retinopathy are reaching more and more people around the world as life expectancy is increasing, which can lead to severe conditions up to total blindness if not treated properly. Unfortunately, there is a global lack of effective treatments for these diseases, as the eye is a complex organ associated with many biological static, dynamic and metabolic barriers that make drug delivery and drug development extremely difficult.

Intravitreal drug delivery has become an efficient and frequently used technique for treating posterior eye afflictions. According to this treatment technique, a drug is typically directly injected into the vitreous humor, also referred to as ‘vitreous body’ of an eye, either of humans or any other vertebrates. Some innovative drug delivery strategies such as implants or microparticles are also developed, but they need reliable models to be tested pre-clinically. Because of the relatively small volume of the vitreous (about 4 mL) that should remain globally constant, the possible injection volume for intravitreal drugs is usually lower than 100 μL. At the moment of the administration, the injected drug is therefore affected by rapid dilution and pH and temperature changes, which can potentially destabilize the API and lead to its aggregation. Moreover, since most of stabilising agents in drug formulations have a very low molecular weight, they are cleared extremely fast compared to large protein drugs, so that the drug is rapidly exposed without stabilizing agents to the stressed conditions of the vitreous humor, where it can interact with all the various components of the vitreous humor.

In general, the difficulties in administering drugs to the posterior part of the eye result from the inherent complexity of this organ, consisting in many static, dynamic and metabolic barriers that consistently limit the diffusion and convection of active pharmaceutical ingredients in eye tissues, thereby impacting their bioavailability. By injecting the drug directly into the vitreous humor, some of these biological barriers are selectively overcome. As such, intravitreal drug delivery methods usually enable that a high fraction of the administered dose of unchanged drug can reach the intended posterior parts of the eye, thereby contributing to a high bioavailability of the ophthalmological drug.

Despite avoiding many of the biological barriers of the eye discussed above, a drug applied intravitreally still has to make its way through remaining barriers and clearance routes.

A first barrier is the vitreous humor itself which significantly affects and limits drug diffusion and convection. The vitreous humor is a complex biological fluid mainly composed of water with additional positively charged collagen and negatively charged proteoglycans. Along with these, many other compounds can be found at low concentrations. The interaction between collagen and the proteoglycans creates a gel-like, rigid structure. As a result of this composition, small molecule drugs usually diffuse more easily than large molecules into this rigid matrix. Further, positively charged molecules usually have significantly lower diffusion rates due to their interaction (and usually aggregation) with the negatively charged proteoglycans. The described vitreous humor composition can vary, e.g., with age, in pathological states, etc., and therewith also its diffusion characteristics.

A second barrier lies in the diffusion of the drug into other parts of the eye, which cause a clearance of the drug from the vitreous humor before it reaches an intended posterior eye part. These dynamic barriers are constituted, e.g., by anterior flow pathway in which vitreous flows and undergoes mass exchange with the aqueous humor during which the drug is cleared by aqueous outflow. Further, small lipophilic drug molecules may also be cleared through the retina-choroid-sclera pathway by diffusing through the retina pigment epithelium and being cleared through circulation of the blood-retinal barrier. Drugs that are substrates of efflux pumps can also be cleared by active transport through the retina-choroid-sclera pathway. In addition, diffusion and convection can be induced by the saccadic movements of the eye which can govern the distribution direction of the drug.

All of the above together results in complex interactions between the vitreous humor components and the applied drugs, with a decisive impact on the drugs' physicochemical stability, bioavailability and hence therapeutic efficacy. Selection of an effective drug formulation and dose is also critical to ensure that the drug remains stable as long as possible and achieves an intended therapeutic effect.

Therefore, the evaluation of the interactions between substances to be applied intravitreally and the vitreous humor (i.e., the physiological environment of the eye) that may affect the drugs' bioavailability are crucial for the development of effective eye affliction treatments.

For analyzing the behavior of substances, various ocular models, i.e. in vitro models, are known for simulation of interactions of said substance with the physiological environment of the eye. These models are mostly static models, which typically are not capable of adequately modelling the above-described dynamic diffusion and convection phenomena after intravitreal injection. For example, such a static model is known from an article of S. Patel et al. with the title “Prediction of intraocular antibody drug stability using ex-vivo ocular model” published in the European Journal of Pharmaceutics and Biopharmaceutics, 2017 March; 112:177-186.

Further, a dynamic model is known from an article of S. Patel et al. with the title “Evaluation of protein drug stability with vitreous humor in a novel ex-vivo intraocular model” published in the European Journal of Pharmaceutics and Biopharmaceutics, 2015 September; 95(Pt B):407-417. The dynamic model comprises three compartments separated by diffusion controlling membranes. Specifically, a vitreous humor compartment is enclosed by a gel matrix compartment which are placed together in a flow-through compartment which is utilized as a buffer reservoir to ensure maintenance of a desired pH level of vitreous humor received in the vitreous humor compartment.

### SUMMARY OF THE INVENTION

It is an object of the present invention to provide an improved method for simulating and analyzing behavior of a substance in an eye, which enables an improved simulation of diffusion and convection procedures that occur in the physiological environment of an eye. Further, it is an object of the present invention to define a method of providing a sample fluid resembling the vitreous humor for use in the above method. Still further, it is an object to provide a buffer fluid and an apparatus for simulating the behavior of a substance in an eye for use in the above method.

These objects are solved by the subject matter of the independent claims.

### BRIEF DESCRIPTION OF THE DRAWINGS

The present disclosure will be more readily appreciated by reference to the following detailed description when being considered in connection with the accompanying drawings in which:

FIG. 1 is a schematic view of an apparatus for testing the behavior of substances in a simulated physiological environment of an eye, wherein various sample cells are used in parallel;

FIG. 2 is a schematic sectional view of a flow cell unit employed in the apparatus depicted in FIG. 1, wherein the flow cell unit houses the sample cell;

FIG. 3 shows a flow diagram illustrating a method for simulating and analyzing behavior of a substance in an eye in which the apparatus depicted of FIGS. 1 and 2 is used;

FIGS. 4 and 5 show flow diagrams illustrating individual steps of the method depicted in FIG. 3;

FIG. 6 shows a diagram illustrating the change of the pH value of a sample over time;

FIG. 7 shows a diagram illustrating the change of a total protein concentration of the sample over time; and

FIG. 8 shows a table illustrating the change of color and turbidity of the sample over time.

### DETAILED DESCRIPTION

Provided herein is an improved method for simulating and analyzing the behavior of a substance in an eye. The method for simulating and analyzing behavior of a substance in an eye comprises the steps of
a) providing at least one sample cell having a void in its inside and a wall delimiting the void, the void is called sample chamber, the wall being at least partly a semi-permeable membrane;
b) filling said sample chamber with a sample comprising at least one substance and a sample fluid constituting a simulated physiological environment of an eye;
c) guiding a flow of buffer fluid, in particular a continuous flow of a buffer fluid, over the outer surface of said semi-permeable membrane for a predetermined period of time; and
d) analyzing the sample.

In general, the sample cell is intended to provide structural conditions allowing to mimic or simulate the physiological conditions within an eye. For doing so, the sample cell has a void in its inside, i.e., a sample chamber for receiving the sample to be analyzed, and a wall delimiting the void, the wall being at least partly a semi-permeable membrane.

The semi-permeable membrane has two surfaces, an inner surface towards the sample cell which forms at least part of the inner surface of the wall delimiting the sample chamber and is thereby in contact with the sample, and an outer surface which is in contact with the buffer fluid.

In the method of the invention, the structural characteristics of the sample cell may be set in accordance with the physiological conditions of an eye for properly simulating physiological conditions of an eye.

Accordingly, the volume of the sample chamber of said sample cell may be provided in accordance with or substantially corresponding to a volume of the eye, the physiological environment of which is to be simulated. ‘Substantially corresponding’ in this context is meant to include the volume of the eye to be simulated as well as deferring therefrom, for example by up to 20% or 30% or 40% or 50%. As a reference, the volume of a human eye is around 4 mL. Accordingly, the volume of the sample chamber may be in the range of 3 mL to 6 mL or in the range of 4 mL to 6 mL or in the range of 4 mL to 5 mL. Particularly, the volume of the sample chamber may be 5 mL or about 5 mL. Generally speaking, the volume of the sample chamber may be greater, in particular slightly greater, than a volume of the eye, the physiological environment of which is to be simulated. The sample chamber of the sample cell may be provided, e.g., by virtue of a dialysis tube or cassette, such as a Float-A-Lyzer® dialysis device. In this configuration, a semi-permeable membrane forms at least a part of the wall delimiting the sample chamber, with the inner surface of the semi-permeable membrane and optionally the wall delimiting the sample chamber, and the outer surface of the semi-permeable membrane and optionally the wall delimiting the sample cell.

In general, semi-permeable membranes are used to control diffusion phenomena. As such, the term “semi-permeable membrane” refers to a diffusion controlling membrane and thus may also be considered a molecular weight size selective membrane. The semi-permeable membrane may be a dialysis membrane. A dialysis membrane is a semi-permeable film, for example a sheet of regenerated cellulose or cellulose esters, containing various sized pores. Generally, molecules larger than the pores cannot pass through the semi-permeable membrane, while molecules smaller than the pores can do so freely. The separation characteristic determined by the pore size-range of the semi-permeable membrane is referred to as the molecular weight cut off (MWCO) of the membrane.

Alternatively or additionally, the structural and functional configuration of the semi-permeable membrane of the sample cell may be provided in accordance with the physiological conditions of the eye to be simulated. Specifically, the molecular weight cut off (MWCO) of the semi-permeable membrane may be provided in accordance with or substantially corresponding to the Retinal Exclusion Limit (REL) of the eye to be simulated. ‘Substantially corresponding’ in this context is meant to include the REL as well as MWCO values deferring from the REL, for example by up to about 30% or 40% or 50%. The REL is generally known as the maximum size of molecules capable of freely diffusing across the retina of an eye. For a healthy human eye, the REL is defined as being in a range of about 50 kDa (10³ Daltons) to 100 kDa, preferably 70 kDa. However, this range may significantly vary, in particular depending on the status of the eye (alteration due to age, decease, etc.). In addition, the REL is highly dependent on the structure of a diffusing molecule, for example, on a linear or globular structure of the molecule. Accordingly, the MWCO of the semi-permeable membrane of the sample cell may be in the range of 50 kDa to 100 kDa, in particular 70 kDa or about 70 kDa.

According to step b) of the present method, the sample chamber is filled with a sample comprising at least one substance and a sample fluid constituting a simulated physiological environment of an eye.

In the context of the present disclosure, the term “substance” is intended to refer to any substance whose behavior in the eye, in particular, in the vitreous humor is of interest. As such, the term “substance” refers to any active agent designed or intended to be applied to or into the eyes of a patient for a therapeutic treatment of an eye disease, in particular posterior eye afflictions, or for alleviating symptoms associated therewith. The substance may be a small molecule or a macromolecule such as a peptide or protein. The term “substance” may further refer to diagnostic agents. The term “substance” may further refer to excipients that are used to formulate active agents or diagnostic agents. The term “substance” may also refer to formulations of said active or diagnostic agent(s). Overall, the term “substance” is not limited to the above but may refer to any substance that may get in contact with the eye, or the vitreous humor, or may be of interest to be tested for its behavior in the physiological environment of an eye.

Preferably, the “substance” is an active agent for treating eye diseases, in particular posterior eye afflictions. The substance may be an active agent for topical, systemic, intravitreal, intrathecal, subcutaneous, subconjunctival, retrobulbar, or intracameral administration. More preferably, the active agent is an intravitreal active agent that is intended to be injected into the vitreous humor of an eye, particularly into the vitreous humor of humans and other vertebrates. Such active agents may be used to treat various eye diseases, such as age-related macular degeneration, diabetic retinopathy, infections inside the eye such as endophthalmitis, etc. For example, the active agent may be or include a monoclonal antibody, in particular a monoclonal antibody in a sterile formulation.

The term “simulated physiological environment of an eye” or “simulated physiological eye environment” as used herein refers to a fluid simulating a natural fluid or condition of an eye, particularly of humans and other vertebrates. As such, the simulated physiological environment may be or comprise a fluid extracted from human or other vertebrate eyes, in particular vitreous humor. For example, the simulated physiological eye environment may comprise or be provided based on porcine vitreous humor. Alternatively or additionally, the simulated physiological eye environment may be an artificial fluid simulating such a natural fluid or condition of the eye.

In the method of the invention, the “sample fluid” is or simulates the physiological environment of the vitreous humor of an eye. In other words, the sample fluid may be a fluid having structural and/or functional characteristics which are equal or correspond to those of a natural fluid or condition of an eye. The sample fluid may be a fluid extracted from human's or other vertebrate's vitreous humor, i.e., extracted vitreous humor, or a mixture of fluids, i.e., it may be provided by mixing or combining fluids. For example, the sample fluid may comprise a first fluid extracted from human's or other vertebrate's vitreous humor, i.e., extracted vitreous humor, and a second fluid constituting a buffer solution. The buffer solution may be identical to the buffer fluid as defined herein below. It is, however, preferred that the sample fluid is a fluid extracted from human's or other vertebrate's vitreous humor, i.e., extracted vitreous humor. Specifically, step b) of the present method may comprise a first sub-step of filling the sample fluid into the sample chamber, and a second sub-step of feeding or injecting said at least one substance into the sample chamber. These sub-steps may be performed in any order. It is, however, preferred that the first sub-step of filling the sample fluid into the sample chamber is performed prior to the second sub-step of feeding or injecting said at least one substance into the sample chamber.

Alternatively, the at least one substance and the sample fluid may be mixed to provide the sample prior to filling the sample into the sample chamber.

The sub-step of feeding or injecting the at least one substance into the sample chamber may be performed such that the typical volume of said at least one substance to be injected into the sample chamber may be in the range between 1 to 200 microliters, such as 2 to 100 microliters, or 10 to 100 microliters. The amount of substance will typically correspond to the amount of substance that is applied to the eye for therapeutic or diagnostic purposes.

As set forth above, the sample fluid constituting the simulated physiological environment may either be an extracted vitreous humor, or a mixture of a first fluid, which may be an extracted vitreous humor and second fluid, which is a buffer solution. Accordingly, the sub-step of filling the sample chamber with the sample fluid may comprise filling a first fluid, e.g., an extracted vitreous humor, and optionally a second fluid, e.g., a buffer solution, into the sample chamber. This may be performed subsequently in any order, or simultaneously. Alternatively, the first and the second fluids, i.e., the extracted vitreous humor and the buffer solution may be mixed prior to the step of filling the sample fluid into the sample chamber.

The buffer solution may be any suitable buffer known to the skilled artisan. It is, however, preferred that the buffer used for preparing the buffer solution of the sample fluid is identical to the “buffer fluid” as defined herein, i.e., identical to the buffer fluid that is guided over the outer surface of the semi-permeable membrane delimiting, at least partly, the sample chamber accommodating the sample to be analyzed.

According to a further development, the method may further comprise a step of providing the sample fluid. The sample fluid may be provided in the form of a gel matrix. Specifically, this step may include sub-steps, in a particular a sub-step of extracting vitreous humor from at least one, preferably more than one isolated vertebrate eyes, specifically from isolated porcine eyes. The extraction may be performed by means of a syringe, e.g., a needle-less syringe. Specifically, the syringe may be inserted in the at least one eye. Thereafter, the syringe may be used to aspirate out the vitreous humor from the eye. For allowing proper insertion of the syringe, the eyeball may be opened, in particular by a slit spaced apart of the iris of the eye, for example about at least 3 mm or 1 cm from the iris. From each eye, e.g., porcine eye, a volume of up to 2 mL or 3 mL of vitreous humor may be extracted. By limiting the amount of extracted vitreous humor to 2 mL or 3 mL per eye, excessive contamination of the extracted vitreous humor, e.g., by tissue cells, may be effectively prevented. The thus extracted vitreous humor may thereafter be pooled and/or cooled, for example with ice or in a fridge, specifically at a temperature of about 5° C.

In a further optional sub-step, the extracted vitreous humor may be subjected to a centrifugation step, wherein the extracted vitreous humor is centrifuged, in particular by using a centrifuge, to separate a clear fraction of the extracted vitreous humor, in particular from cell debris. Thereafter the clear fraction may be extracted and isolated, e.g., by being aspirated with a pipette.

In an alternative or additional optional sub-step, the extracted vitreous humor may be filtered, in particular by applying pressure filtration. Specifically, for doing so, a pressure filtration unit may be used with a polyether sulfone filters, e.g., having a pore size of about 0.10 to 0.30 μm, preferably of about 0.15 to 0.25 μm, more preferably of about 0.20 to 0.24 μm, in particular of 0.22 μm.

In an alternative or additional optional sub-step, the extracted vitreous humor then may be frozen, in particular at a temperature of about −80° C., prior to being further processed or used in the method.

In an alternative or additional optional sub-step, the extracted vitreous humor may be combined with a buffer solution so as to provide the sample fluid. The buffer solution may be identical to or different from the buffer fluid, but is preferably identical.

According to step c) of the present method set forth above, a flow of a buffer fluid, also referred to as ‘buffer fluid flow’ herein, is guided over the outer surface of the semi-permeable membrane delimiting, at least partly, the sample chamber accommodating the sample to be analyzed.

The buffer fluid, which is guided over the surface of the semi-permeable membrane may the identical to, or different from the buffer solution that is used as a component of the sample fluid in the sample chamber, preferably it is identical.

Preferably, the ‘buffer fluid flow is a continuous flow. During step c), the flow of buffer fluid is continuously guided over the outer surface of the semi-permeable membrane for a predetermined period of time, also referred to as ‘testing time’ herein. The testing time may refer to a time period of one or more hours, but may also be lower than one hour. Specifically, the testing time may be in the range of 4 h to substantially a multiple of 24 h. For example, the testing time may be a time period of 4 h or 24 h or 72 h or 96 h or 168 h or 336 h or 504 h. Further, the buffer fluid flow may have a constant flow rate. As set forth above, by guiding the buffer fluid flow over the outer surface of the semi-permeable membrane, the outer surface of the sample cell may be continuously provided with fresh buffer fluid, thereby providing dynamic conditions around the sample cell. Preferably, the flow of buffer fluid is provided such that a laminar flow of buffer fluid is guided over the semi-permeable membrane. In this way, a homogenous and optimal diffusion via the semi-permeable membrane may be achieved, which allows to simulate the natural environment of an eye, in particular the dynamic and metabolic barriers prevailing therein.

According to one configuration, the flow rate of buffer fluid flow may be a constant flow rate. Further, the flow rate may be in the range of 5 mL/min to 12 mL/min, particularly in the range of 6 mL/min to 10 mL/min, and more particularly about 8 mL/min

More preferably, the buffer fluid is provided such that, upon being guided over the outside surface of the semi-permeable membrane, it sets or affects the desired conditions of the sample contained in the sample chamber, in particular a predetermined pH condition of the sample received in the sample chamber. In a specific embodiment, it further sets or affects at least one of a predetermined temperature condition and a predetermined osmolality condition of the sample received in the sample chamber. Alternatively or additionally, the buffer fluid may, upon being guided over the outer surface of the semi-permeable membrane, receive or absorb substances which diffuse out of the sample chamber over the semi-permeable membrane into the buffer fluid, in particular precipitation or degradation products from the sample contained in the sample chamber.

Accordingly, in the context of the present disclosure, the term ‘buffer fluid’ refers to a buffer which has a desired pH value and/or is able to maintain a pH value of the sample received in the sample chamber, for example between pH 5.5 and pH 8.5, specifically between about pH 7.0 and about pH 7.6, more particularly about pH 7.4.

Specifically, the buffer fluid may comprise salts. Preferably, the buffer fluid is an aqueous buffer fluid. The buffer fluid may for example be a phosphate buffered saline, a bicarbonate buffer, Ringer's bicarbonate buffer, Ringer's lactate buffer, simulated body fluids, other isotonic solutions, cell culture medias, and any other physiologically representative buffers. Ringer's lactate buffer, also known as Ringer's lactate solution (RL), also known as sodium lactate solution and Hartmann's solution, is a mixture of sodium chloride, sodium lactate, potassium chloride, and calcium chloride in water.

The buffer fluid may also be used as the buffer solution for the preparation of the sample fluid which may be provided in the form of a gel matrix.

According to one configuration, the buffer fluid is an artificial substitute for vitreous humor, e.g., for human vitreous humor. The buffer fluid may comprise at least one cationic species selected from the group of cations of sodium, potassium, calcium and magnesium and at least one anionic species selected from the group of chloride, bicarbonate, phosphate, lactate and optionally a preservative such as azide. The buffer fluid may comprise one, two, three or all four of the respective cationic and anionic species.

The buffer fluid may further comprise glucose and/or glutathione disulfide.

Specifically, the buffer fluid may comprise sodium, potassium, calcium, magnesium, chloride, bicarbonate, phosphate, lactate, glucose and/or glutathione disulfide, and optionally a preservative such as azide.

Exemplary salts for the preparation of the buffer fluid include sodium chloride, potassium chloride, dibasic sodium phosphate, sodium bicarbonate, calcium chloride dihydrate, magnesium chloride hexahydrate, sodium lactate and sodium azide. The buffer fluid may comprise one, two, three, four, five, six, seven, or all of these salts.

In one embodiment, the buffer fluid comprises (A) a buffer comprising sodium chloride, potassium chloride, calcium chloride dihydrate, magnesium chloride hexahydrate, dibasic sodium phosphate and sodium bicarbonate, (B) a buffer comprising sodium chloride, potassium chloride, calcium chloride dihydrate and sodium lactate, and (C) sodium azide.

In one embodiment, the buffer fluid comprises (A) a buffer comprising sodium chloride, potassium chloride, calcium chloride dihydrate, magnesium chloride hexahydrate, dibasic sodium phosphate, sodium bicarbonate, and glucose, (B) a buffer comprising sodium chloride, potassium chloride, calcium chloride dihydrate and sodium lactate, and (C) sodium azide.

In one embodiment, the buffer fluid comprises (A) a buffer comprising sodium chloride, potassium chloride, calcium chloride dihydrate, magnesium chloride hexahydrate, dibasic sodium phosphate, sodium bicarbonate, glucose and glutathione disulfide, (B) a buffer comprising sodium chloride, potassium chloride, calcium chloride dihydrate and sodium lactate, and (C) sodium azide.

An example for (A) above is commercially available BSS PLUS® Sterile Intraocular Irrigating Solution (for example from Alcon). An example for (B) above is commercially available Ringer Lactate Solution (for example from B. Braun).

In a preferred embodiment, the buffer fluid comprises
between about 50 mmol/L and about 250 mmol/l sodium,
between about 1.0 mmol/L and about 25 mmol/L potassium,
between about 0.2 mmol/L and about 5.0 mmol/L calcium,
between about 0.1 mmol/L and about 4.0 mmol/L magnesium,
between about 25 mmol/L and about 200 mmol/L chloride,
between about 5 mmol/L and about 100 mmol/L bicarbonate,
between about 0.1 mmol/L and about 10 mmol/L phosphate,
between about 0.8 mmol/L and about 20 mmol/L lactate,
between about 0.8 mmol/L and about 20 mmol/L glucose,
between about 0.1 mmol/L and about 10 mmol/L glutathione disulfide,
   and optionally a preservative, e.g., between about 0.5 mmol/L and about 10 mmol/L azide.

Specifically, the buffer fluid may comprise
between about 100 mmol/L and about 200 mmol/l sodium,
between about 2.0 mmol/L and about 10 mmol/L potassium,
between about 0.4 mmol/L and about 2.5 mmol/L calcium,
between about 0.2 mmol/L and about 2.0 mmol/L magnesium,
between about 75 mmol/L and about 175 mmol/L chloride,
between about 10 mmol/L and about 50 mmol/L bicarbonate,
between about 0.1 mmol/L and about 5.0 mmol/L phosphate,
between about 1.5 mmol/L and about 15 mmol/L lactate,
between about 1.5 mmol/L and about 15 mmol/L glucose
between about 0.1 mmol/L and about 5.0 mmol/L glutathione disulfide,
   and optionally a preservative, e.g., between about 1.0 mmol/L and about 5.0 mmol/L azide.

More specifically, the buffer fluid may comprise
between about 125 mmol/L and about 175 mmol/l sodium,
between about 3.0 mmol/L and about 7.0 mmol/L potassium,
between about 0.6 mmol/L and about 1.5 mmol/L calcium,
between about 0.5 mmol/L and about 1.3 mmol/L magnesium,
between about 100 mmol/L and about 150 mmol/L chloride,
between about 10 mmol/L and about 40 mmol/L bicarbonate,
between about 0.1 mmol/L and about 4.0 mmol/L phosphate,
between about 1.5 mmol/L and about 8.0 mmol/L lactate,
between about 2.0 mmol/L and about 8.0 mmol/L glucose,
between about 0.1 mmol/L and about 4.0 mmol/L glutathione disulfide,
   and optionally a preservative, e.g., between about 1.5 mmol/L and about 4.5 mmol/L azide.

Yet more specifically, the buffer fluid may comprise
between about 140 mmol/L and about 160 mmol/l sodium,
between about 4.5 mmol/L and about 6.0 mmol/L potassium,
between about 0.9 mmol/L and about 1.3 mmol/L calcium,
between about 0.6 mmol/L and about 1.0 mmol/L magnesium,
between about 120 mmol/L and about 130 mmol/L chloride,
between about 17 mmol/L and about 25 mmol/L bicarbonate,
between about 0.2 mmol/L and about 3.0 mmol/L phosphate,
between about 3.0 mmol/L and about 5.0 mmol/L lactate,
between about 2.0 mmol/L and about 5.0 mmol/L glucose,
between about 0.2 mmol/L and about 3.0 mmol/L glutathione disulfide,
   and optionally a preservative, e.g., between about 2.5 mmol/L and about 3.5 mmol/L azide.

In a particularly preferred embodiment, the buffer fluid comprises
about 155.6 mmol/L±10% sodium,
about 5.1 mmol/L±10% potassium,
about 1.1 mmol/L±10% calcium,
about 0.9 mmol/L±10% magnesium,
about 127.3 mmol/L±10% chloride,
about 21.2 mmol/L±10% bicarbonate,
about 2.5 mmol/L±10% phosphate,
about 4.3 mmol/L±10% lactate,
about 4.2 mmol/L±10% glucose,
about 2.5 mmol/L±10% glutathione disulfide,
   and optionally a preservative, e.g., about 3.0 mmol/L±10% azide.

In a more particularly preferred embodiment, the buffer fluid comprises
about 155.6 mmol/L±5% sodium,
about 5.1 mmol/L±5% potassium,
about 1.1 mmol/L±5% calcium,
about 0.9 mmol/L±5% magnesium,
about 127.3 mmol/L±5% chloride,
about 21.2 mmol/L±5% bicarbonate,
about 2.5 mmol/L±5% phosphate,
about 4.3 mmol/L±5% lactate,
about 4.2 mmol/L±5% glucose,
about 2.5 mmol/L±5% glutathione disulfide,
   and optionally a preservative, e.g., about 3.0 mmol/L±5% azide.

In a most particularly preferred embodiment, the buffer fluid comprises
about 155.6 mmol/L sodium,
about 5.1 mmol/L potassium,
about 1.1 mmol/L calcium,
about 0.9 mmol/L magnesium,
about 127.3 mmol/L chloride,
about 21.2 mmol/L bicarbonate,
about 2.5 mmol/L phosphate,
about 4.3 mmol/L lactate,
about 4.2 mmol/L glucose
about 2.5 mmol/L glutathione disulfide,
   and optionally a preservative, e.g., about 3.0 mmol/L azide.

The osmolality of the freshly prepared buffer fluid is preferably between about 280 mOsm/kg H2O and about 330 mOsm/kg H₂O, more preferably between about 300 mOsm/kg H₂O and about 315 mOsm/kg H₂O and most preferably between about 305 mOsm/kg H₂O and about 310 mOsm/kg H₂O.

In accordance with the method of the present invention, the buffer fluid is conditioned by subjecting the buffer fluid to an atmosphere of a predetermined CO₂ concentration for a predetermined period of time, before and/or during guiding the buffer fluid flow over the outer surface of the semi-permeable membrane. In a specific embodiment, the conditioning further comprises subjecting the buffer fluid to a predetermined temperature for a predetermined period of time, before and/or during guiding the buffer fluid flow over the outer surface of the semi-permeable membrane.

The step of conditioning may be performed such that the buffer fluid is subjected to an atmosphere of predetermined conditions, such as an atmosphere of a predetermined CO₂ concentration and optionally a predetermined temperature. Said conditioning may be done for a predetermined period of time. For doing so, the buffer fluid, may be accommodated in an incubator configured to generate an atmosphere of predefined conditions. By doing so, characteristic of the buffer fluid may be set at desired conditions and/or maintained stable. For example, the conditioning step may prevent the buffer fluid from being subjected to an unintended pH shift.

The conditioning may be done once or continuously, for example once before the buffer fluid is used, or continuously during use of the buffer fluid. For example, the buffer fluid may be subjected to continuous conditioning while it is guided in a flow, in particular in a continuous flow, over the outer surface of the semi-permeable membrane to an atmosphere of predetermined conditions, such as an atmosphere of a predetermined CO₂ concentration and optionally a predetermined temperature.

For example, the buffer fluid may be subjected to an atmosphere having a temperature of about 37° C. and a CO₂ concentration of about 7%. For doing so, the buffer fluid may be accommodated in an incubator configured to generate an atmosphere of predefined conditions for the predetermined period of time. As set forth above, the conditioning may be performed for a predetermined period of time which may also be referred to as ‘conditioning time’ herein. The conditioning time may be set to allow for proper conditioning of the buffer fluid. The conditioning time may be one or more hours. Specifically, the conditioning time may be at least 2 h or 6 h or 8 h. For example, the conditioning may be performed overnight, i.e. for at least 6 h. Advantageously, the buffer fluid is conditioned in a CO₂ incubator prior to and during use at a temperature between about 35° C. and 40° C. and a CO₂ concentration between about 5% and 10%, preferably at a temperature of about 37° C. and a CO₂ concentration of about 7%.

In a specific embodiment, the buffer fluid resembles the human vitreous humor. This means that the properties of the buffer fluid are similar, or essentially identical, to the properties of human vitreous humor. Said properties include the type and concentration of anionic and cationic species and preferably further include pH, osmolality and viscosity.

The inventors have observed that the buffer fluid is likely to undergo microbial contamination during long-term stability studies, despite sterile filtration, as it is highly nutritive. This can be prevented by the addition of a preservative. Azide salts, such as sodium azide have been found to efficiently suppress microbial growth in the buffer.

The buffer fluid of the present invention is advantageous in that it is standardized, easy to prepare and to handle, while closely reflecting the conditions in vitreous humor, in particular human vitreous humor.

In the context of the present invention, it has been surprisingly found that dynamic conditions within and at an outer surface of said sample chamber may be assured by guiding a flow of buffer fluid (preferably conditioned as outlined above), in particular a continuous, laminar flow of such a buffer fluid, over the outer surface of a semi-permeable membrane at least partially delimiting said sample chamber. The laminar flow of buffer fluid accommodates a stable diffusion process of the at least one substance within the sample cell and the semi-permeable membrane interface. This allows to more adequately simulate the natural environment of an eye, in particular the dynamic and metabolic barriers prevailing therein. As a result, the suggested method provides an improved in vitro model allowing to analyze the behavior of a substance in the eye more realistically, without the need to revert to invasive treatments.

In general, the proposed method refers to an in vitro method. The proposed method may be conducted using components of an organism that have been isolated from their usual biological surroundings.

The proposed method allows to assess the behavior of substances in a simulated physiological environment of an eye, particularly of humans and other vertebrates. As such, the method may be used to simulate, to determine, or allow predictions, e.g., on how a substance is affected over time by the biological environment in the eye, particularly by the biological environment in the vitreous humor. The potential impact on the intended therapeutic effect may also be determined on this basis.

To that end, the method may be applied, but is not limited, to analyze and assess the stability of a substance in a simulated physiological eye environment. Such analyses may refer to, e.g., concentration dependent precipitation of substances. Further, the method may be used to determine interactions of substances, such as for example macromolecules and/or excipients in a specific fluid environment such as the vitreous humor, for example with proteins in the vitreous humor; the stability of a substance upon dilution in a specific fluid, for example vitreous humor, and after loss of stabilizing excipients, for example surfactants, sugars, buffering species and tonicity agents; and long term stability in simulated physiological eye environment, in particular in the vitreous humor.

According to step d) the present method further comprises analyzing the sample. For doing so, the sample may be removed from the sample chamber prior to performing the step of analyzing the sample. In one embodiment, the method may further comprise a step of separating said at least one substance from the sample fluid, such as for example, separating a protein active agent from the sample fluid, before performing the step of analyzing.

Specifically, the step of analyzing the sample may comprise determining the pH value of the sample, in particular at a predetermined temperature, e.g. 37° C., of the sample. This may be performed by a pH-meter.

Alternatively or additionally, a total protein concentration of the sample may be measured, in particular by spectrophotometry, e.g. at about 280 nm.

Alternatively or additionally, osmolality of the sample may be determined, for example by a freezing point depression method.

Alternatively or additionally, the step of analyzing the sample may comprise measuring or determining a physicochemical stability of the substance in said sample, such as an active agent contained therein. Specifically, this step may be performed to analyze degradation, denaturation and aggregation of the at least one substance. For doing so, the step of analyzing physicochemical stability of the sample may comprise visual inspection to determine the presence or density of visible particles in the sample, e.g., the visual inspection under a black and white box, or under a low magnification.

Alternatively or additionally, the presence and density of sub-visible particles may be determined, e.g. by light obscuration, or by flow imaging microscopy techniques (like Micro-flow imaging, or FlowCA. etc).

Alternatively or additionally, a color of the sample may be determined, e.g., by visual comparison with color standards, or by colorimetry etc. Alternatively or additionally, turbidity of the sample may be determined, e.g. by a plate based or cuvette based spectrophotometry, or by performing turbidimetry techniques, etc. Alternatively or additionally, aggregation and oligomerization of the at least one substance in the sample may be determined, e.g. by gel permeation chromatography, or by size-exclusion chromatography, in particular by ultraviolet-visible spectroscopy, e.g. at about 280 nm, or by fluorescent spectroscopy.

Alternatively or additionally, the step of analyzing the physicochemical stability of the sample may comprise determining a total substance concentration, e.g., the total protein concentration, by methods known in the art, such as spectrophotometry, e.g. at about 280 nm, or by surface plasmon resonance (SPR), or by any other method known for this purpose by the skilled artisan. Further, the step of analyzing the sample may be performed to measure or determine chemical purity and/or chemical stability of the sample. In this way, chemical modifications and degradations, in particular of the at least one substance, may be determined. Specifically, chemical purity may be determined by any method known in the art, e.g., by size-exclusion chromatography, in particular by ultraviolet-visible spectroscopy, e.g., at about 280 nm. Alternatively or additionally, charge variance may be determined, e.g. by anion exchange chromatography, or by cation exchange chromatography, in particular with ultraviolet-visible spectroscopy detection at 280 nm.

Alternatively or additionally, a sub-unit analysis may be performed, e.g. by capillary electrophoresis under denaturing and non-denaturing conditions. Other suitable methods including the ones outlined above are known to the skilled artisan.

In a further development, for analyzing the behavior of a substance, the method may further comprise a step of analyzing the buffer fluid, in particular after being guided over the outer surface of the semi-permeable membrane. This step may be performed during the testing time, while the outer surface of the semi-permeable membrane is subjected to the flow of buffer fluid and/or thereafter. In other words, the step of analyzing the buffer fluid may be performed in parallel to the step of analyzing the sample and/or parallel to the step of guiding the flow of buffer fluid over the outside surface of the semi-permeable membrane. This step may be applied when the apparatus is provided with a closed buffer fluid circuit or with an opened buffer fluid circuit.

Specifically, the step of analyzing the buffer fluid may comprise determining a pH value of the buffer fluid, in particular at a predetermined temperature thereof, e.g. 37° C. This may be performed, e.g., by a pH-meter. Alternatively or additionally, a total protein concentration in the buffer fluid may be measured, in particular by spectrophotometry, e.g. at about 280 nm or other suitable methods commonly known for this purpose. Alternatively or additionally, osmolality of the buffer fluid may be determined, for example by a freezing point depression method, or by measuring the conductivity, or by other commonly known analytic methods.

Alternatively or additionally, the step of analyzing the buffer fluid may comprise measuring or determining degradation, denaturation and aggregation products of the at least one substance (e.g., a protein drug such as a mAb) in the buffer fluid. For doing so, the step of analyzing physicochemical stability of the buffer fluid may comprise visual inspection to determine the presence or density of visible particles therein. Alternatively or additionally, the presence and density of sub-visible particles may be determined, e.g. by light obscuration. Alternatively or additionally, a color of the buffer fluid may be determined, e.g. by colorimetry. Alternatively or additionally, turbidity of the buffer fluid may be determined, e.g. by performing turbidimetry techniques. Alternatively or additionally, aggregation and oligomerization products of the substance in the buffer fluid may be determined, e.g. by size-exclusion chromatography, in particular by ultraviolet-visible spectroscopy, e.g. at about 280 nm. Alternatively or additionally, denaturation products of the substance may be determined, e.g. by micro differential scanning calorimetry. Alternatively or additionally, the step of analyzing physicochemical stability of the substance may comprise determining a total protein concentration, in particular by spectrophotometry, e.g. at about 280 nm.

All of the above mentioned analytical methods are commonly known; other methods known by the skilled artisan for the above analytical purposes may also be used.

### Conditioning

According to a further development, the method may further comprise a step of conditioning the sample fluid before adding the at least one substance. Specifically, the sample fluid may be conditioned prior to being filled into the sample chamber, but it is equally preferred that the sample fluid is conditioned after being filled into the sample cell. The step of conditioning may be performed such that the sample cell containing said sample fluid is subjected to an atmosphere of predetermined conditions, such as an atmosphere of a predetermined CO₂ concentration. The step of conditioning may further comprise subjecting the sample cell containing said sample fluid to a predetermined temperature. Said subjecting may be done for a predetermined period of time. For example, the sample fluid, preferably said extracted vitreous humor, may be subjected to an atmosphere having a CO₂ concentration of about 7% and optionally a temperature of about 37° C. For doing so, sample cell containing the sample fluid, preferably said extracted vitreous humor, may be accommodated in an incubator configured to generate an atmosphere of predefined conditions for the predetermined period of time. As set forth above, the conditioning may be performed for a predetermined period of time which may also be referred to as ‘conditioning time’ herein. The conditioning time may be set to allow for proper conditioning of the sample fluid. The conditioning time may be one or more hours. Specifically, the conditioning time may be at least 2 h or 6 h or 8 h. For example, the conditioning may be performed overnight, i.e. for at least 6 h.

Alternatively or additionally, the conditioning of the sample fluid, preferably of said extracted vitreous humor, may be conveniently effected by virtue of using the buffer fluid that has been conditioned as outlined above, also as the buffer solution used for forming the sample fluid. Alternatively, or additionally, the sample fluid, preferably said extracted vitreous humor, may be conditioned after being filled into the sample chamber, by guiding the conditioned buffer fluid flow over the outer surface of the semi-permeable membrane during the testing, and optionally, also for a predetermined time before the testing.

In the context of the present invention, it has been found that the step of conditioning the sample fluid, in particular the extracted vitreous humor, has the effect that the pH value of the sample fluid, e.g. of the extracted vitreous humor, in particular porcine vitreous humor, remains constant or is more stable during testing. In other words, a pH level of a conditioned sample fluid, in particular of a conditioned extracted vitreous humor, may be more stable when performing the method compared to an unconditioned sample fluid.

The sub-step of feeding or injecting the substance into the sample chamber may be performed after the step of conditioning the sample fluid as outlined above.

### Labelling

According to a further development, the method may comprise a step of labelling said at least one substance, e.g., with a fluorescent dye. If the at least one substance is a pharmaceutical formulation of an active agent with pharmaceutically acceptable excipients, it is sufficient that said active agent is labelled, e.g., with a fluorescent dye. By doing so, the step of analyzing the sample may be performed more effectively. This is because the determination of the purity of the at least one substance in the sample may be more accurately assessed, e.g. by using fluorescent detection, and more accurately distinguished from proteins in the sample, e.g. from porcine vitreous humor proteins.

The method may comprise a further step of conditioning the substance, optionally after labelling it (e.g., with the fluorescent dye), prior to being fed into the sample chamber. For doing so, the substance, may be subjected to an atmosphere of predetermined conditions, i.e. of a predetermined CO₂ concentration an optionally a predetermined temperature, e.g. at about 7% CO₂ concentration and optionally about 37° C., said subjecting may be done for a predetermined period of time, i.e. the conditioning time as described herein, also with all its embodiments. The conditioning the substance may be performed prior to feeding it into the sample chamber.

### Sample Cells Used in Parallel

In a further development, the method disclosed herein may be performed such that more than one sample cell is provided and filled with the sample as described above. Each one of the sample cells may subjected to the flow of buffer fluid as described above in parallel or simultaneously to any other sample cell. The step of guiding the flow of buffer fluid over the semi-permeable membranes at least partially delimiting the sample chambers of the different sample cells may be performed such that each sample cell is subjected to the flow of buffer fluid for an individual period of time, i.e., an individual testing time. For example, when providing a first and a second sample cell which are filled with a sample, the first sample cell may be subjected to the flow of buffer fluid for a first period of time and the second sample cell may be subjected to a second period of time which differs from the first period of time.

Further, the step of analyzing the samples may be performed such that the different samples, when being analyzed, have been placed in their associated sample cell for the different individual periods of time, in parallel or simultaneously to each other. For example, when filling a first sample in the first sample chamber and a second sample in the second sample chamber, the first sample is analyzed after being placed for the first period of time in the first sample chamber during which the first sample cell has been exposed to the flow of buffer fluid. Accordingly, the second sample is analyzed after being placed for the second period of time in the second sample chamber during which the second sample cell has been exposed to the flow of buffer fluid. By this configuration, analysis at different points in time are enabled, while preventing analyzing the same samples and sample cells several times which would disrupt the osmotic equilibrium within the sample cells. In other words, in this way, the method allows to simultaneously test and analyze a plurality of different samples, which is performed in that each sample cell is subjected to the flow of buffer fluid for an individual period of time (Tj) which may differ among the sample cells.

### Sample Fluid, Method for Providing and Use of a Sample Fluid

Provided herein is a sample fluid, prepared by extracting vitreous humor from vertebrate eyes; centrifuging the extracted vitreous humor to separate a clear fraction of the extracted vitreous humor; and filtering the extracted vitreous humor, and optionally, conditioning the extracted vitreous humor by subjecting said extracted vitreous humor to an atmosphere of a predetermined CO₂ concentration for a predetermined period of time. In one embodiment, said extracted vitreous humor may further be subjected to a predetermined temperature for a predetermined period of time.

Further, a method for providing a sample fluid for use in the method as described above is provided. Specifically, the method for providing a sample fluid comprises the steps of extracting vitreous humor from vertebrate eyes; centrifuging the extracted vitreous humor to separate a clear fraction of the extracted vitreous humor; and filtering the extracted vitreous humor, and optionally, conditioning the extracted vitreous humor by subjecting the said extracted vitreous humor to an atmosphere of a predetermined CO₂ concentration for a predetermined period of time. In one embodiment, said extracted vitreous humor may be subjected to a predetermined temperature for a predetermined period of time. Since the method for providing the sample fluid is intended to be used in the above described method, technical features which are described in connection with the method for simulating and analyzing behavior of an ophthalmological substance in an eye may also relate and be applied to the method for providing a sample fluid or to the sample fluid, and vice versa.

Further provided is a sample fluid that is obtainable by the method for providing a sample fluid described hereinabove.

Also provided herein is the use of a sample fluid described herein in the method for simulating and analyzing the behavior of a substance in an eye as described hereinabove.

### Buffer Fluid and Use of a Buffer Fluid

Still further, a buffer fluid is provided, wherein the buffer fluid is composed as described hereinabove, and optionally, also conditioned as described above. Further provided is a use of a buffer fluid in the methods described herein is provided, wherein the buffer fluid is composed as described hereinabove, and optionally, also conditioned as described above. In one embodiment, said buffer fluid comprises sodium, potassium, calcium, magnesium, chloride, bicarbonate, phosphate, lactate, glucose and optionally a preservative such as azide.

Since the buffer fluid is intended to be used in the above described method, technical features which are described in connection with the method for simulating and analyzing behavior of an ophthalmological substance in an eye may also relate and be applied to the method for providing a sample fluid, and vice versa.

### Apparatus

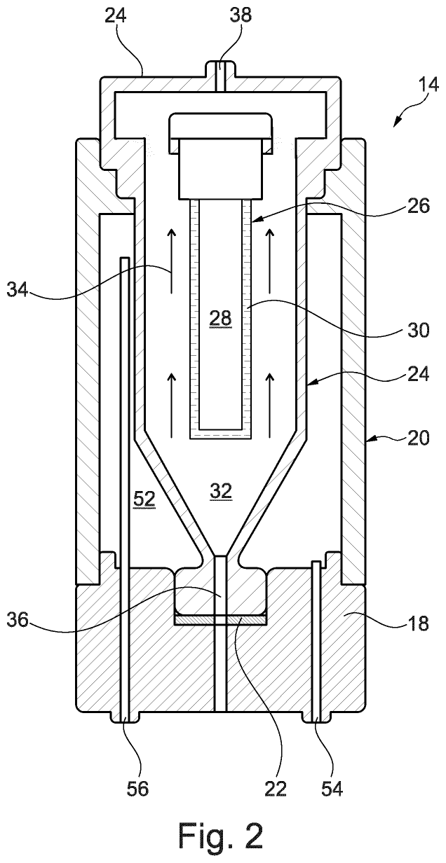

As apparent from FIG. 2, the sample cell may be placed, in particular, may be removably mounted, in a buffer cell having a void in its inside which is called flow chamber. The flow chamber is flown through by the buffer fluid, i.e., the flow of buffer fluid. According to one configuration, the buffer cell has a flow chamber, a buffer fluid inlet configured for feeding the flow chamber with the buffer fluid, i.e., for guiding the flow of buffer fluid into the flow chamber of the buffer cell, and a buffer fluid outlet configured for discharging the buffer fluid after being guided through the flow chamber, i.e., for directing the flow of buffer fluid out of the flow chamber of the buffer cell. Thereby, the flow chamber fluid-communicatively connects the buffer fluid inlet to the buffer fluid outlet.

Specifically, the sample cell may be placed in the flow chamber of the buffer cell. According to this configuration, the sample cell may entirely or partly be placed within the flow chamber. At least, the semi-permeable membrane of the sample cell may be placed within the flow chamber. By such a configuration, the buffer fluid, subsequently, is directed into the flow chamber, flows through the flow chamber while being guided over the outer surface of the semi-permeable membrane, and thereafter is discharged from the flow chamber, thereby generating the continuous flow over the semi-permeable membrane.

The buffer cell, in particular the structural arrangement of the buffer fluid inlet, buffer fluid outlet and flow chamber, may be designed such that the continuous flow of buffer fluid being guided through the buffer cell flows along an outer surface of the semi-permeable membrane of the sample cell, thereby the buffer fluid that is present in the buffer cell is continuously replaced. According to one configuration, the flow rate of buffer fluid through the buffer cell may be a constant flow rate. The flow rate may be in the range of 5 mL/min to 12 mL/min, particularly in the range of 6 mL/min to 10 mL/min, and more particularly about 8 mL/min.

In a further development, the buffer cell, that is the inlet and the outlet of the buffer cell, may be connected to a buffer fluid circuit. The buffer fluid circuit may be a closed buffer fluid circuit.

According to this configuration, buffer fluid discharged from the buffer cell may be processed, e.g., filtered and conditioned as described above, before being redirected through the buffer cell. For doing so, the closed buffer fluid circuit may be provided with a filter, e.g., a fiber filter, which may be arranged downstream of the buffer cell. In the context of the present disclosure, the term ‘downstream’ and ‘upstream’ refers to a flow direction of the flow of buffer fluid and a position before or after the sample cell respectively. The filter may be configured to filter the buffer fluid after being guided through the buffer cell. By doing so, clogging of aggregated degradation products, which have been received by the buffer fluid upon flowing through the buffer cell, may be prevented. The filter may be a glass fiber filter, in particular, having a pore size of 1 μm. Alternatively or additionally, the closed buffer circuit may comprise a buffer reservoir into which buffer fluid discharged from the buffer cell, in particular after being filtered, may be fed and stored before being redirected into the buffer cell.

Alternatively, the buffer cell may be connected to an open buffer fluid circuit. In this configuration, the buffer fluid discharged from the buffer cell may not be redirected again into the buffer cell, but rather collected and stored in a discharge container. Fresh buffer fluid may be fed from a buffer reservoir into the buffer cell.

In one configuration, the sample cell 26 comprising the sample chamber 28, and the buffer cell 24 comprising the flow chamber 32, a buffer fluid inlet 36 and a buffer fluid outlet 38 are arranged such that the sample chamber 28 and the flow chamber 32 are in direct contact with each other through and separated from each other by the semi permeable membrane 30. In this configuration, the buffer fluid inlet 36 is configured for feeding the flow chamber 32 with the buffer fluid, i.e., for guiding the flow of buffer fluid 34 into the flow chamber 32 of the buffer cell 24, and the buffer fluid outlet 38 is configured for discharging the buffer fluid 34 after being guided through the flow chamber 32, i.e., for directing the flow of buffer fluid 34 out of the flow chamber 32 of the buffer cell 24. Thereby, the buffer fluid communicatively connects the buffer fluid inlet to the buffer fluid outlet. The buffer fluid is directed into the flow chamber, flows through the flow chamber while being guided over the outer surface of the semi-permeable membrane, and thereafter is discharged from the flow chamber, thereby generating the flow (e.g., a continuous flow) over the semi-permeable membrane.

Since the apparatus 10 is intended to be used in the above-described method for simulating and analyzing behavior of an ophthalmological substance in an eye, technical features which are described in connection with said method, in particular technical features pertaining to the flow of buffer fluid, to the sample cell, sample chamber, buffer cell and flow chamber or to the buffer fluid may also relate and be applied to the apparatus, and vice versa.

### Use of an Apparatus

Furthermore, a use of an apparatus in the above described method for simulating and analyzing behavior of an ophthalmological substance in an eye is provided. Specifically, the apparatus is configured for stimulating behavior of an ophthalmological substance in an eye and comprises at least one sample cell having a sample chamber which is delimited by a semi-permeable membrane, wherein the sample cell is configured to receive a sample comprising an ophthalmological substance and a sample fluid constituting a simulated physiological environment of an eye; and a buffer supply system configured to guide a flow of buffer fluid over the outside surface of the semi-permeable membrane for a predetermined period of time (Tⱼ).

Since the apparatus is intended to be used in the above described method, technical features which are described in connection with the method for simulating and analyzing behavior of an ophthalmological substance in an eye may also relate and be applied to the method for providing a sample fluid, and vice versa.

### DETAILED DESCRIPTION OF PREFERRED EMBODIMENTS

In the following, the invention will be explained in more detail with reference to the accompanying Figures. In the Figures, like elements are denoted by identical reference numerals and repeated description thereof may be omitted in order to avoid redundancies.

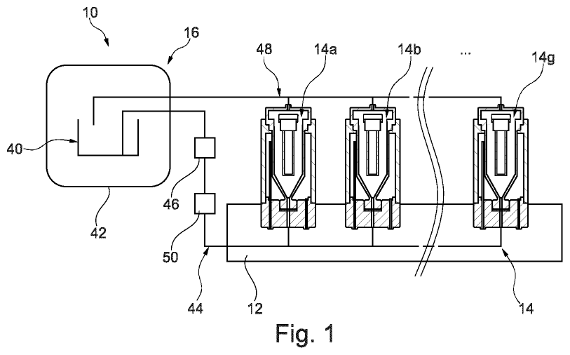

FIG. 1 shows an apparatus 10 for simulating behavior of a substance in an eye. The apparatus comprises a base 12 accommodating a plurality of flow cell units 14. While the shown apparatus 10 comprises seven flow cell units 14a to 14g, the apparatus is not limited to this number and accordingly may comprise more or less than seven flow cell units 14. For the sake of better visualization, only the three flow cell units 14a, 14b and 14g are shown, wherein the other four flow cell units are indicated by curved lines. Preferably, the different flow cell units 14 are structurally identical.

The apparatus 10 further comprises a buffer supply system 16 configured to supply the plurality of flow cell units 14 with buffer fluid.

FIG. 2 shows an enlarged view of one exemplary flow cell unit 14 of the apparatus 10 which, for the purpose of better visualization, is depicted isolated from the other parts of the apparatus 10. In the following, characteristics of the flow cell unit 14 are described exemplarily based on the flow cell unit 14 depicted in FIG. 2 which may apply correspondingly to any one of the different flow cell units 14 of the apparatus 10.

A flow cell unit 14 comprises a cell mount 18 which is designed to removably mount the flow cell unit 14 to the base 12 of the apparatus 10. Specifically, the cell mount 18 is configured to be firmly mounted to an upper part of the base 12, i.e. by a screw connection, where it remains during normal operation of the apparatus 10. The flow cell unit 14 further comprises a casing 20 having a hollow-cylindrical form. The casing 20, at a bottom end section, is connected together with the cell mount 18 as one component.

The upper part of the cell mount 18 is provided with a centrally arranged recess in which a flat gasket 22 is inserted. A buffer cell 24 is inserted in the center of the casing 20 such that a bottom end face of the buffer cell 24 sits on the gasket 22 in the recess of the cell mount 18, as can be gathered from FIG. 2. Thereby, the buffer cell 24 is connected directly to gasket 22 as one component. The contact surface between a top end part of the buffer cell 24 and a top end part of the casing 20 may be provided with at least one further gasket (not shown) to ensure a watertight connection there-between. Also connecting surfaces between a bottom part of the casing 20 and the upper part of the cell mount 18 may be provided with a gasket (not shown) to ensure a sealed connection.

In the center of the buffer cell 24, a sample cell 26 is inserted such that the sample chamber 28 as well as the semi-permeable membrane 30 delimiting the sample chamber 28 are fully arranged within the buffer cell 24. In an alternative configuration, the sample cell 26 may also be entirely arranged within the buffer cell 24 and may, for example, be provided in the form of a floating cell.

The sample cell 26 is configured to receive a sample, the behavior of which is to be tested during operation of the apparatus 10. For doing so, the sample cell 26 is preferably submerged in the buffer cell 24. The connection between the sample cell 26 and the buffer cell 24 is a sealed connection, which can be realized, e.g., by the corresponding threads or by a further gasket (not shown). Specifically, the sample cell 26 may be provided in the form of or comprise a dialysis tube or cassette, such as Float-A-Lyzer® dialysis device. The buffer fluid outlet 38 may be located on the screw cap.

The flow cell unit 14 is provided with three different chambers or compartments which are specified in the flowing.

The sample cell 26 is provided with a sample chamber 28 which is delimited, at least partly, by a semi-permeable membrane 30. In the shown configuration, the sample chamber 28 has a cylindrical shape, at least the cylindrical surface and the bottom surface of the cylinder is provided by the semi-permeable membrane 30. Specifically, the sample chamber 28 has a volume as described herein, also with all its embodiments.

The buffer cell 24 is provided with a flow chamber 32 in which the sample cell 26 is arranged such that the semi-permeable membrane 30 delimiting the sample chamber 28 is entirely received in the flow chamber 32. The flow chamber 32 is delimited by outer walls of the buffer cell 24 and configured to be flown through with a buffer fluid. In other words, the flow chamber 32 constitutes a buffer flow channel, through which a buffer fluid flow 34, preferably a laminar flow, is directed as indicated by arrows in FIG. 2. For generating the buffer fluid flow 34 in the flow chamber 32, the buffer cell 24 comprises a buffer fluid inlet 36 for supplying the buffer fluid into the flow chamber 32 and a buffer fluid outlet 38 for discharging buffer fluid from the flow chamber 32 after being guided there-through. The buffer fluid inlet 36 and the buffer fluid outlet 38 are fluid-communicatively connected to the buffer supply system 16 shown in FIG. 1 which is configured to direct buffer fluid into the buffer fluid inlet 36 and to discharge buffer fluid after being guided through the flow chamber 32 form the buffer fluid outlet 38. As can be gathered from FIG. 2, the cell mount 18 and the gasket 22 are provided with an inlet channel via which the buffer fluid inlet 36 is fluid-communicatively connected to the buffer supply system 16.

As shown in FIG. 1, the buffer supply system 16 constitutes a closed buffer fluid circuit. As such, the buffer supply system 16 comprises a buffer reservoir 40 for storing and conditioning buffer fluid to be directed to the buffer cells 24. Specifically, the buffer reservoir 40 is arranged in an incubator 42 configured to subject the buffer reservoir 40, i.e. the buffer fluid stored therein, to a predetermined atmosphere at a predetermined CO₂ concentration and optionally at a predetermined temperature, with the predetermined CO₂ concentration and the predetermined temperature as described herein, also with all their embodiments. A buffer supply line 44 is provided having a supply manifold which directs buffer fluid to the different flow cell units 14, respectively. For doing so, a pump unit 46 is arranged in the supply line 44 upstream of the supply manifold and configured to selectively and variably generate a continuous and constant flow through the flow chambers 32 of the different flow cell units 14. Specifically, in one operating mode, the buffer supply system 16 is operated such that buffer fluid flows through each flow chamber 32 of the different flow cell units 14 at a flow rate as described herein, also with all its embodiments. The buffer fluid, before being channeled to the buffer cell units 14, is filtered in a filter unit 50, e.g. comprising a glass fiber filter having a pore size as described herein, also with all its embodiments, before being redirected into the buffer reservoir 40. Further, a buffer discharge line 48 is provided having a discharge manifold which discharges and collects buffer fluid from the different flow cell units 14, respectively.

Further, as can be gathered from FIG. 2, a tempering chamber 52 is provided between the casing 20 and the buffer cell 24 which is configured to guide a tempering medium, e.g. conditioned water having a predetermined temperature, e.g., of about 37° C., over the outer surface of the buffer cell 24. In this way, a temperature of the buffer cell 24 and thereby of the sample cell 26 may be set and maintained at a desired temperature. For doing so, the casing 20 is provided with a tempering medium inlet 54 and a tempering medium outlet 56 designed such that the tempering medium, upon being fed into the tempering chamber 52 via the tempering medium inlet 54, is guided around the outer surface of the buffer cell 24 before being discharged via the tempering medium outlet 56. For supplying the tempering chamber 52 with the tempering medium, the apparatus 10 is provided with a tempering system (not shown) configured to supply the tempering chamber 52, with tempering medium via the tempering medium inlet 54 and to discharge tempering medium from the tempering chamber 52 via the tempering medium outlet 56. The tempering system may further comprise a heat exchanger unit configured to provide heat exchanging between the buffer fluid and the tempering medium, in particular before guiding the buffer fluid into the flow chamber 32.

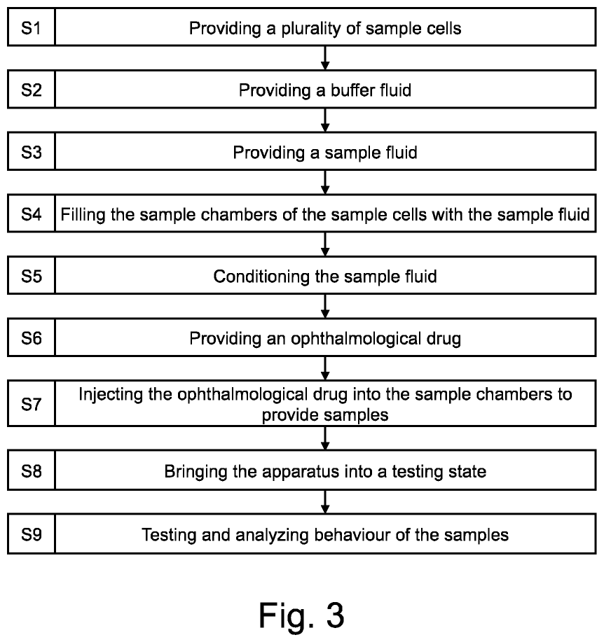

In the following, under reference to FIG. 3, a method for simulating and analyzing behavior of a substance in an eye is described which makes use of the apparatus 10 depicted in FIGS. 1 and 2. Specifically, the method is applied to test and analyze behavior, e.g., of a substance in vitreous humor, in particular human vitreous humor, i.e., in a simulated eye environment.

In a first step S1 of the method, the apparatus 10 is provided which comprises the plurality of flow cell units 14, each of which is equipped with one sample cell 26 having the sample chamber 28 which is at least partly delimited by the semi-permeable membrane 30.

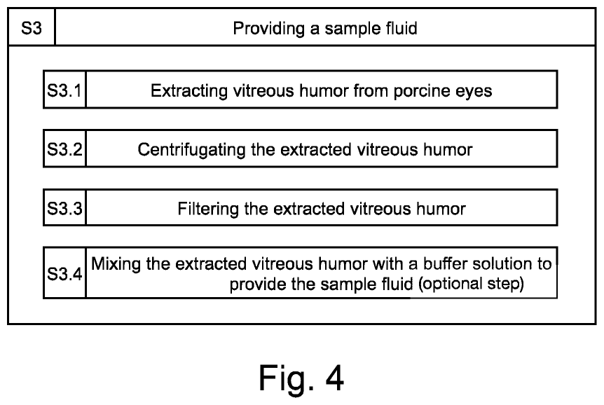

In a subsequent step S2 the buffer fluid is provided which is stored in the buffer reservoir 40. The buffer fluid is a buffer fluid composed as described herein, also with all its embodiments. In a step S3, a sample fluid is provided which is intended to simulate vitreous humor of a human eye, and which is preferably an extracted vitreous humor. This is performed in several sub-steps as shown in FIG. 4. In a first sub-step S3.1, porcine vitreous humor is extracted from a plurality of porcine eyes. The extracted vitreous humor is then centrifuged in sub-step S3.2. Specifically, the extracted vitreous humor is placed in a centrifuge tube of a centrifuge in which it is centrifuged, e.g., for 10 to 30 min at 3,000 to 11,000 rpm, for 10 to 20 min at 3,900 rpm or for 20 min at 10,000 rpm.

By doing so, a clear fraction of the extracted vitreous humor is separated from, e.g., cell debris and thereafter extracted, e.g., by being aspirated with a pipette. The extracted clear fraction of the vitreous humor is then filtered in sub-step S3.3, e.g., by a pressure filtration unit, in particular having a polyether sulfone filters with a pore size as describe herein, also with all its embodiments. Optionally, the filtered vitreous humor may be frozen for a predetermined time prior to being further processed. Thereafter, in an optional sub-step S3.4, the vitreous humor is mixed with a buffer solution, thereby providing the sample fluid. The buffer solution may be identical to the buffer fluid.

In a step S4, the sample chamber 28 of each flow cell unit 14 is filled with the sample fluid. For doing so, the sample cell 26 of each flow cell unit 14 may be disassembled from its associated flow cell unit 14 and the top part of the sample cell 26 is opened. After filling the sample chambers 28 of the sample cells 26 with the sample fluid, the sample cells 26 are closed by installing the top part of the sample cell 26, and each of the sample cells 26 are then housed within the respective buffer cell 24.

A step S5 may be performed after filling the sample into the sample cells 26 prior to housing the sample cell 26 into the respective buffer cell 24. This is performed by placing each sample cell 26 accommodating the sample fluid in an incubator, which may be the incubator 42, for a predetermined period of time, i.e., the above described conditioning time, e.g. for 6 h. In this way, the sample fluid is subjected to an atmosphere of a predetermined CO₂ concentration and optionally a predetermined temperature for a predetermined period of time.

In optional step S6, a substance, in particular an intravitreal active agent, is provided. This step may be performed simultaneously with step S4 or S5. Specifically, during S6, the substance may be provided and optionally pre-labeled, e.g., with a fluorescent dye. The thus labeled substance may then be conditioned by subjecting it to an atmosphere of a predetermined CO₂ concentration and optionally a predetermined temperature for a predetermined period of time. For doing so, the substance is placed into an incubator, which may be the incubator 42, for a predetermined period of time. Additionally, or alternatively, the conditioning of the substance may be achieved by guiding the conditioned buffer fluid 32 over the outer surface of the semi-permeable membrane 30 after step S7, or both.

Then, in step S7, the substance is injected into each sample chamber 28 accommodating the sample fluid. In this way, the sample cells 26 are provided with the samples which are to be tested and analyzed.

The sample cells 26 prepared for performing the testing, i.e., simulating of the behavior of the sample accommodated therein, are then placed in the associated flow cell units 14, thereby bringing the apparatus 10 in a testing state in which it is ready to perform testing (step S8). Thereafter, the behavior of the samples is tested and analyzed in step S9 which is described in the following under reference to FIG. 5. In this step, a total number J of individual flow cell units 14, each flow cell unit 14 containing one sample cell 26 with one sample, are subjected to simulated testing conditions. Accordingly, the integer J indicates the total number of flow cell units 14, i.e., seven flow cell units 14 in the shown configuration, of which each sample chamber 28 comprises a sample of same composition and condition at the beginning of the test. The integer j, however, refers to one specific of the J different flow cell units 14, or sample cells 26, or samples in the sample chambers 28, and thus has a value between 1 and J. The testing is performed such that the different sample cells are tested, i.e. subjected to a simulated condition, for different and predetermined periods of time Tⱼ, i.e. different testing times. Accordingly, the parameter Tⱼ refers to the testing duration of the j^{th} sample cell 26, wherein this predetermined time differs among the different sample cells 26. The different testing times Tⱼ may have a duration as described herein, also with all its embodiments. Specifically, the Tⱼ may be defined as follows:

Tⱼ=Tⱼ−1+Dⱼ, 1≤j≤J (1)

wherein Dⱼ refers to a period of time greater 0 which may be constant, i.e. each one of D₁ to D_{J} may have the same value, or which may vary, i.e. D₁ to D_{J} may have different values. In the present configuration, e.g., D₁ equals 0, D₂ equals 4 h, D₃ equals 20 h, D₄ equals 72 h, D₅ equals 72 h, D₆ equals 168 h and D₇ equals 168 h.

In a first sub-step S9.1, the variable j is set to equal 1. The following sub steps are performed as long as the variable j has not passed the number J of sample cells 26 used as indicated by S9.2. Then, in sub-step S9.3, the apparatus 10 is operated such that buffer fluid is flown around each sample cell 26 in that the flow 34 of buffer fluid is guided over the outer surface of the semi-permeable membrane 30 of each sample cell 26. Specifically, this step is performed in such a manner that a laminar flow 34 of buffer fluid is guided over the outer surface of the semi-permeable membrane 30. By doing so, the buffer fluid sets a predetermined pH condition, a predetermined temperature condition and a predetermined osmolality condition of the samples received in the different sample chambers 28. Further, the buffer fluid flowing over the semi-permeable membrane 30 of the sample cells 26 receives any substances which diffuse form the sample through the semi-permeable membrane into the buffer fluid, such as, e.g., precipitation or degradation products from the sample received in the sample chamber.

This sub-step is performed as long as the parameter t indicating the time elapsed from beginning of the flow of the buffer fluid in sub-step S9.3 has not reached Tⱼ as indicated by S9.4. If t has reached Tⱼ, the method proceeds to sub-step S9.5 in which the j^{th} sample cell 26 is removed from the apparatus 10. During this sub-step S9.5, the flow of buffer fluid 34 may be interrupted or continued.

Thereafter, in sub-step S9.6, the removed j^{th} sample cell 26 is analyzed, e.g. by determining a physicochemical stability of the sample. In other words, the sample received in the j^{th} sample cell 26 is analyzed after subjecting the semi-permeable membrane 30 of the j^{th} sample cell 26 to the flow of buffer fluid 34 for the predetermined period of time Tⱼ. The step of analyzing the sample received in the sample cell 26 may be performed as described herein, also with all its embodiments. At the same time, the variable j is incremented by 1 in sub-step 9.7 and the method returns to sub-step S9.2, thereby repeating sub-steps S9.3 to 9.7 until all sample cells 26 have been removed and analyzed.

Accordingly, in the method disclosed herein, more than one sample cell 26 is provided and its sample chamber 28 filled with a sample, wherein the step of guiding the flow 34 of buffer fluid over the semi-permeable membrane 30 of the different sample cells 26 is performed such that each sample cell 26 is subjected to the flow 34 of buffer fluid for the individual period of time Tⱼ which differs among the sample cells 26.

In a further development, the method may further comprise a step of analyzing the buffer fluid, in particular after being guided over the outer surface of the semi-permeable membrane 30. This step may be performed in parallel to step 9.6 and/or in parallel to step 9.3. The step of analyzing the buffer fluid may be performed as described herein, also with all its embodiments.

### EXAMPLE

In the following, a specific configuration of the method is described by way of an example.

### Materials:

Porcine eyes were obtained from by a local slaughterhouse in Basel (Switzerland)
The drug products (monoclonal antibodies (mAbs)) were provided by Lonza AG
BSS PLUS® Irrigating Solution (cat. #0008000048) was purchased from Alcon (Fort Worth, USA): BSS PLUS® is a sterile intraocular irrigating solution composed of two parts. Part I is a sterile 480 mL solution in a 500 mL single-dose bag to which the Part II concentrate is added. Each mL of Part I contains: sodium chloride 7.44 mg, potassium chloride 0.395 mg, dibasic sodium phosphate 0.433 mg, sodium bicarbonate 2.19 mg, hydrochloric acid and/or sodium hydroxide (to adjust pH), in water for injection. Part II is a sterile concentrate in a 20 mL single-dose vial for addition to Part I. Each mL of Part II contains: calcium chloride dihydrate 3.85 mg, magnesium chloride hexahydrate 5 mg, glucose 23 mg, glutathione disulfide (oxidized glutathione) 4.6 mg, in water for injection.
After addition of BSS PLUS® solution Part II to the Part I bag, each mL of the reconstituted product contains: sodium chloride 7.14 mg, potassium chloride 0.38 mg, calcium chloride dihydrate 0.154 mg, magnesium chloride hexahydrate 0.2 mg, dibasic sodium phosphate 0.42 mg, sodium bicarbonate 2.1 mg, glucose 0.92 mg, glutathione disulfide (oxidized glutathione) 0.184 mg, hydrochloric acid and/or sodium hydroxide (to adjust pH), in water for injection.
The reconstituted product has a pH of approximately 7.4. Osmolality is approximately 305 mOsm.
Ri-lac nach Hartmann solution (cat. #FV10452) was purchased from BBraun (Sempach, Switzerland). 1000 mL of the solution contain 6.0 g Sodium Chloride, 0.40 g potassium chloride, 0.27 g calcium chloride-dihydrate, 6.24 g sodium lactate as a sodium lactate solution (50% w/w, corresponding to 3.12 g Sodium lactate), water; (concentration of electrolyte mmol/l: Na⁺ 131, K⁺ 5.4, Ca²⁺ 1.8, Cl⁻ 112, lactate-Ions 28); pH: 5.0-7.0;
theoretical osmolarity=277 mOsm/L.
Float-A-Lyzer G2 Dialysis Device MWCO 50 kDa 5 mL (cat. #G235058) were purchased from Spectrum Laboratories Inc. (USA)
Vivaspin® (6 and 20) centrifugal concentrators with 10 or 30 kDa membrane filters (cat. #Z614483-25EA) were purchased from Sartorius (Steiheim, Germany)
Sodium azide: Sigma-Aldrich (MW 65 g/mol) Material no. 71289
All chemicals were of analytical grade.

### Apparatus:

The test was performed using the ‘USP 4 DFZ II’ dissolution tester of the manufacturer ERWEKA (Flow-Through Cell USP Apparatus 4 dissolution tester), which was configured to receive seven separate flow cell units. The different flow cell units have been customized to receive a Float-A-Lyzer® G2 dialysis device form the manufacturer Spectra-Por as the sample cell having a semi-permeable membrane with a MWCO of 50 kDa and a sample chamber with a volume of 5 mL to mimic the REL of the human eye (around 70 kDa) and its volume (around 4 mL).

### Preparation of the Buffer Fluid (Simulating the Vitreous Humor):

To prevent using large volumes of extracted porcine vitreous humor (PVH), an artificial vitreous humor (VH) buffer was created and used with similar composition to human VH for most of its electrolytes (see Table 1 below). The buffer fluid was obtained by combining two commercially available components: the BSS PLUS® Irrigating Solution and the Ri-lac nach Hartmann solution, to reach the concentrations in Table 1. For this purpose, 1 L of the BSS Irrigating Solution was added to an appropriate beaker, and 180 mL of Ri-Lac solution were added. Subsequently, 0.235 g of sodium azide were weighed and added to the buffer fluid (to give a final concentration of 0.02% w/v or 3.0 mmol/L therein) to prevent microbial growth despite sterile filtration, as the PVH content is highly nutritive for microorganisms and may undergo contamination for long stability studies.

TABLE 1
Composition of the VH buffer compared to human VH

| Compound | Human VH [mmol/L] | VH Buffer [mmol/L] |
|---|---|---|
| Na⁺ | 146.7 | 155.6 |
| K⁺ | 5.7 | 5.1 |
| Ca²⁺ | 1.1 | 1.1 |
| Mg²⁺ | 0.9 | 0.8 |
| Cl⁻ | 121.6 | 127.3 |
| Bicarbonate | 15.0 | 21.2 |
| Phosphate | 0.4 | 2.5 |
| Lactate | 4.0 | 4.3 |
| Ascorbate | 2.0 | — |
| Glucose | 3.0 | 4.2 |

The buffer fluid was conditioned at all times by being subjected (and stored during all the duration of the stability study) in a CO₂ incubator (Fisherbrand®, Thermo Fisher Scientific) at 37° C. and 7% CO₂ to equilibrate the pH to 7.4.

The conditioning of the buffer fluid in the CO₂ incubator at 37° C. and 7% CO₂ to equilibrate the pH to 7.4 prevents the pH to shift as previously observed during PVH incubation where the buffer was not conditioned in this manner.

### Porcine Vitreous Humor Extraction:

The porcine eyes were kept in ice and handled with sterilized material under a laminar flow hood. The eyes were immobilized and incised with a scalpel about 1 cm from the iris (towards the posterior part of the eye) to reach and open the vitreous chamber. Care was taken to extract as much PVH as possible with a needle-less syringe (2 mL) without opening the aqueous chamber (the aqueous humour being a contaminant due to different composition). Despite a theoretical volume of about 4 mL, not more than 2-3 mL have been extracted from the vitreous chamber as too many tissue cell contaminants tended to be aspirated after this volume.

The samples were then pooled and kept at all time in ice or in a fridge at 5° C. The PVH were first centrifuged (20 min at 3900 rpm using an Eppendorf 5810R Refrigerated Centrifuge) to sediment cell or tissue debris. The top fraction were then isolated and further filtered with a Pressure Filtration unit (Milipore) with 0.22 pm disposable polyether sulfone (PES) filters. Due to the high viscosity of PVH, and the high amount of cell debris/tissue contaminations from the dissection process, the filtration process required frequent change of filters.

All the filtrated samples were aliquoted in fractions of 6 mL and frozen at −80° C. until use.

### Preparation of the Float-A-Lyzer® G2 Cassettes:

The Float-A-Lyzer® G2 cassettes (sample cells) had been opened and filled and submerged in a 10% ethanol:water (v:v) solution for 10 minutes. The solution was then removed and the sample chambers were flushed with deionized water.

One day before the start of the stability study run, the PVH samples were thawed and injected (4.5 mL of PVH) into activated Float-A-Lyzer® cassettes (sample cells), which were placed into buffer fluid contained in a beaker, and the beaker was incubated at 37° C. and 7% CO₂ overnight. Specifically, each sample chamber was filled with 4 mL of the extracted PVH at room temperature. For doing so, the extracted PVH was filtered with a syringe (e.g., 18G, silicon oil free) having a syringe filter with a pore size of 0.22 μm before being filled into the sample chambers of the Float-A-Lyzer® cassettes. Then, each sample cell was vertically placed into a beaker filled with the buffer fluid which were placed in the incubator overnight, i.e., at least for 8 h, to subject the buffer and the sample cells to an atmosphere having a temperature of about 37° C. and a CO₂ concentration of about 7%. During this conditioning step, the buffer in the beaker had been stirred.

Thereafter, the content of each sample chamber was checked by determining that the pH of the PVH is in the range of pH 7.2 to 7.4. Further, it was ensured that the protein concentration of the vitreous humor was comparable in the different sample chambers. Usually, if the pH is out of the desired range, the step of conditioning may be repeated for a longer duration and/or at improved stirring and gas exchange conditions, e.g., with higher beaker volume, fresh vitreous humor and/or higher stirring speed. If the protein concentration in one sample chamber is significantly lower compared to the others, this sample chamber may be exchanged by virtue of exchanging the sample cell.

### Stability Study Run:

On the start day (TO), the cassettes (=sample cells) were transferred into the VH-FTC (Flow-Through Cell USP Apparatus 4 dissolution tester) and the monoclonal antibody (mAb) drug was injected (about 3 mg) into each cell as described in Table 2.

For this purpose, 50 μL of monoclonal antibody priorily filtered with a 18G filter needle, was injected into six of the seven sample chambers containing the conditioned PVH (conditioned in the buffer fluid as described above). The time point at which the monoclonal antibody was filled into the sample chamber was marked as TO. Because sampling several times each cell would disrupt the osmotic equilibrium in the cassettes for the following timepoints, each timepoint required using all the content of the cassette. Therefore, at each sampling, the buffer fluid flow was stopped and all the content of the cassette was transferred into glass vials for analysis. The buffer fluid flow rate applied during the testing was 8 mL/min.

TABLE 2
VH-FTC cell time point repartition

| Timepoint | Content |
|---|---|
| T₀ | Drug in PVH |
| T₁ = 4 h | Drug in PVH |
| T₂ = 24 h (1 d) | Drug in PVH |
| T₃ = 96 h (4 d) | Drug in PVH |
| T₄ = 168 h (1 week) | Drug in PVH |
| T₅ = 336 h (2 weeks) | Drug in PVH |
| T₆ = 504 h (3 weeks) | Drug in PVH |

### Analysis:

### Constant pH Over Time

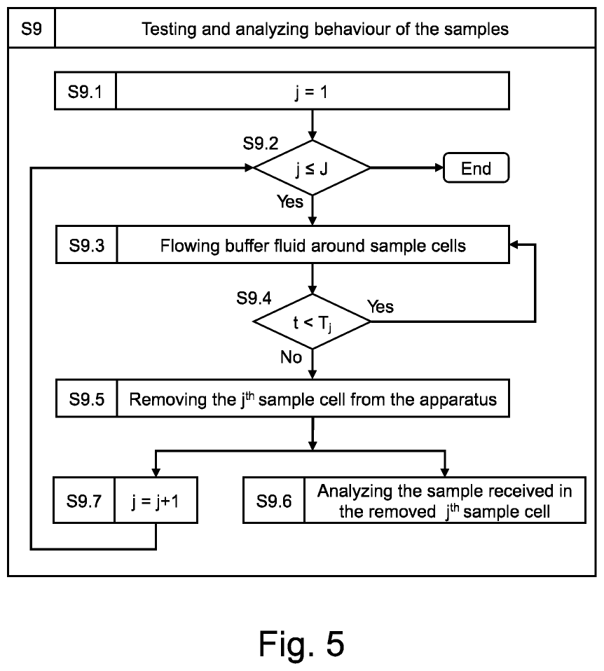

The pH value of the different samples was determined at a predetermined temperature of 37° C. with a pH-meter. The result of the measurement is depicted in FIG. 6, which illustrates the change of the pH value of the sample over time (the time points are shown in table 2). Specifically, the abscissa of the diagram in FIG. 6 refers to the testing time after which the measurement of the corresponding sample was performed, while the ordinate refers to the pH value.

As can be gathered from FIG. 6, with the suggested configuration of the apparatus as described above, the pH value of the sample was maintained in the desired range throughout the testing time, i.e. in a range of 0.5 around the initial value. These pH variations are extremely small compared to the pH shift (moving from more than 1 pH unit) observed by others (see, e.g., Patel et al., Eur J Pharm Biopharm. 2015; 95(Pt B):407-417). It is believed that this favorable result is due to the CO₂ conditioning applied which seems to properly maintain the pH in this configuration.

This shows that the natural environment of an eye was adequately simulated under the conditions applied.

### Total Protein Concentration

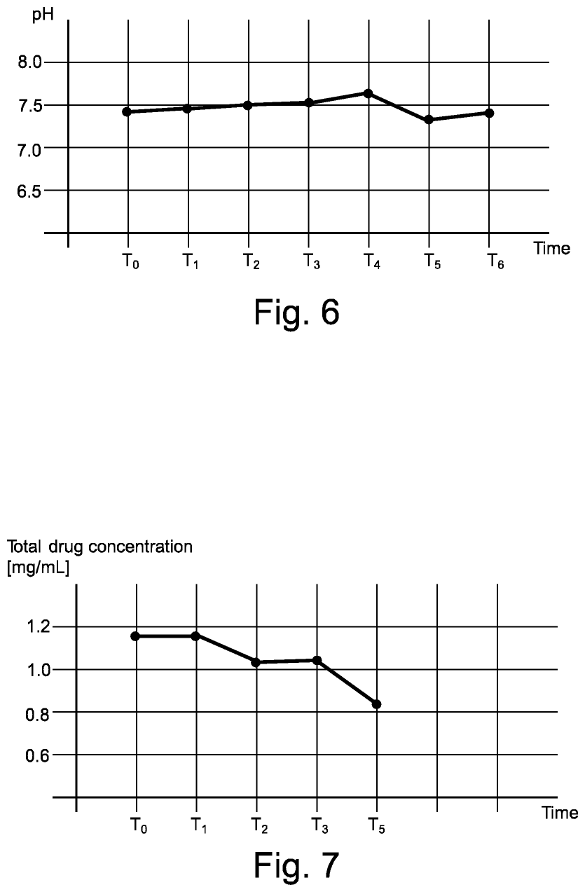

Further, the total protein concentration of the different samples was determined by spectrophotometry. The results are shown in FIG. 7 depicting a diagram which illustrates the change of the protein concentration of the sample over time.

Specifically, the abscissa of the diagram refers to the testing time after which the measurement of the corresponding sample was performed, and the ordinate of the diagram refers to the total drug protein concentration (mg/mL). As can be gathered from FIG. 7, the protein amount showed a gradual decrease in protein content, to a small extend in the first days, and with a more pronounced decrease after 2 weeks.

All the intact drug with associated agglomerates and PVH proteins larger than 50 kDa should remain in the cassette, unless they get degraded into smaller pro-ducts. This was observed in FIG. 7 as the protein amount (from both PVH and drug) decreased over time but remained close to the initial mass (at least for the first week, and is still quite high even after 2 W). This means that some products were flushed away (necessarily degradation products as the initially present LMWS of PVH are removed during the equilibration step), but that most of the drug and PVH remained in the cassette during all that period. The drug can therefore still interact with the PVH proteins for all this period, which should allow this model to effectively maintain the stressed conditions of the PVH environment with related physicochemical changes for weeks. This demonstrates that the present method retains the drug in the dialysis cassette while removing degradation products, and hence allows for properly determining the behavior of the protein drug in a simulated eye environment.

### Assessment of Physicochemical Stability Changes of the mAb Drug Over Time: Colorimetry and Turbidity Measurement

Expected in vivo modifications of the mAb drug (due to physicochemical instability of the mAb) were captured by the present method in vitro by analyzing the aggregation and precipitation behavior by colorimetry and turbidity assessment.

The color of the different samples was determined by colorimetry. The color scale was based on the European Pharmacopeia Colour Standard 2.2.2 Method 1, which consists of 37 reference color standards with encoding. “BY7” stands for “Brown/Yellow standard no 7, and “<” and “>” stand for “more” or less intense than” as the comparison is always made with the closest reference standard with the highest intensity.

The turbidity of the samples was determined by performing turbidimetry techniques. The turbidity was measured in Nephelometric Turbidity Units (NTU).

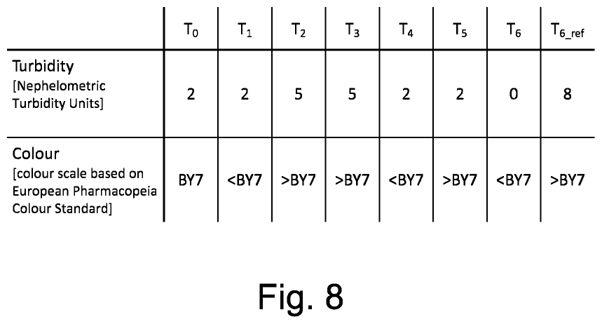

The result of these analyses is shown in FIG. 8 depicting a table which illustrates the change of color and turbidity over time. The first row of the table refers to the turbidity measurement. The second row to the colorimetry measurement after the different testing durations T.

In the table depicted in FIG. 8, the last column refers to a reference sample which is a sample of the vitreous humor, i.e., vitreous humor without the monoclonal antibody, and which has been subjected to the buffer fluid flow for three weeks (T₆).

This demonstrates that the present method is sensitive enough to study in vitro the physicochemical behavior of an intravitreal drug in vivo over time.

### Labelling of the mAb

The mab drug has been labelled with a fluorescent dye (Alexa Fluor™ 488, cat #A20000, Invitrogen™), in a molar mab:dye ratio of 1:4, according to the manufacturer of Alexa Fluor 488. Prior to incubating the drug, unbound dye has been removed by centrifugation (Princeton PRO SPIN, cat #CS-800, Nippon Genetics Europe GmbH). The labelled drug has been injected into the PVH in the VH-FTC and analysed using SEC HPLC with fluorescent detection (excitation at 495 nm/emission at 519 nm). This allowed for an accurate assessment of the purity of the drug, and to distinguish it from PVH proteins.

Stability testing during intravitreal drug development is extremely challenging as the vitreous humor is a complex biological fluid inside a complex organ with many static, dynamic and metabolic barriers, and the in vivo, ex vivo and in vitro models known in the art fail to either properly represent this system or to allow a good assessment of drug stability changes. The above results demonstrate that unlike previous in vitro models, the present in vitro method manages to simulate and analyse the behavior of intravitreal drugs in extracted VH under simulated in vivo conditions.

These exemplary embodiments and items only depict examples of a plurality of possibilities. Hence, the embodiments shown here should not be understood to form a limitation of these features and configurations. Any possible combination and configuration of the described features can be chosen according to the scope of the invention.

### LIST OF REFERENCE NUMERALS

10 apparatus
12 base
14 flow cell unit
16 buffer supply system
18 cell mount
20 casing
22 gasket
24 buffer cell
26 sample cell
28 sample chamber
30 semi-permeable membrane
32 flow chamber
34 flow of buffer fluid
36 buffer fluid inlet
38 buffer fluid outlet
40 buffer reservoir
42 incubator
44 buffer supply line
46 pump unit
48 buffer discharge line
50 filter unit
52 tempering chamber
54 tempering medium inlet 56 tempering medium outlet

### ITEMS OF THE INVENTION

The invention further comprises the following items.
1. Method for simulating and analyzing the behavior of a substance in an eye, comprising the steps of:
   a) providing at least one sample cell (26) having a sample chamber (28) which is at least partly delimited by a semi-permeable membrane (30);
   b) filling the sample chamber (28) with a sample comprising at least one substance and a sample fluid constituting a simulated physiological environment of an eye;
   c) guiding a flow of buffer fluid (34) over the outer surface of said semi-permeable membrane (30) for a predetermined period of time (Tⱼ); and
   d) analyzing the sample.
2. The method according to item 1, wherein, before performing step c), the buffer fluid is conditioned by subjecting the buffer fluid to an atmosphere of a predetermined CO₂ concentration for a predetermined period of time.
3. The method according to items 1 or 2, wherein said flow of buffer fluid in step c) is a laminar flow, preferably, a continuous laminar flow, and/or wherein the buffer fluid flow has a flow rate in the range between 6 mL/min to 10 mL/min, particularly of about 8 mL/min.
4. The method according to any one of the preceding items, wherein the substance is an active agent, preferably an active agent for topical, systemic, intravitreal, intrathecal, subcutaneous, subconjunctival, retrobulbar, or intracameral administration, and wherein the sample fluid is or simulates vitreous humor.
5. The method according to any one of the preceding items, further comprising a step of providing the sample fluid, including the sub-steps of:
   extracting vitreous humor from at least one isolated vertebrate eye;
   centrifuging the extracted vitreous humor to separate a clear fraction of the extracted vitreous humor;
   filtering the extracted vitreous humor; and
   optionally mixing the extracted vitreous humor with a buffer solution to provide the sample fluid.
6. The method according to any one of the preceding items, wherein the sample chamber (28) has a volume in the range of 4 mL to 6 mL, in particular of about 5 mL, and/or wherein the semi-permeable membrane (30) has a molecular weight cut off in the range of 50 kDa to 100 kDa, in particular of about 70 kDa.
7. The method according to any one of the preceding items, further comprising a step of labelling the substance with a fluorescent dye.
8. The method according to any one of the preceding items, wherein the buffer fluid, upon being guided over the semi-permeable membrane (30) of the sample cell (26):
   sets a predetermined pH condition of the sample received in the sample chamber (28); and
   receives substances which diffuse out of the sample chamber (28) over the semi-permeable membrane into the buffer fluid, in particular precipitation or degradation products from the sample received in the sample chamber (28).
9. The method according to any one of the preceding items, wherein the buffer fluid comprises at least one cationic species selected from the group of sodium, potassium, calcium, magnesium, and at least one anionic species selected from the group of chloride, bicarbonate, phosphate, lactate, glucose and optionally a preservative such as azide.
10. The method according to any one of the preceding items, wherein the step of analyzing the sample comprises determining the physicochemical stability of the at least one substance.
11. The method according to any one of the preceding items, wherein more than one sample cell is provided in step a), and wherein the predetermined period of time (Tj) in step c) of guiding a flow of buffer fluid (34) over the outer surface of said semi-permeable membrane (30) of each sample cell (26) is different for the different sample cells (26).
12. Sample fluid prepared by extracting vitreous humor from vertebrate eyes; centrifuging the extracted vitreous humor to separate a clear fraction of the extracted vitreous humor; and filtering the extracted vitreous humor, and optionally, conditioning the extracted vitreous humor by subjecting the said extracted vitreous humor to an atmosphere of a predetermined CO₂ concentration for a predetermined period of time.
13. A method for providing a sample fluid for use in the method according to items 1 to 11, comprising the steps of:
   extracting vitreous humor from vertebrate eyes;
   centrifuging the extracted vitreous humor to separate a clear fraction of the extracted vitreous humor; and
   filtering the extracted vitreous humor, and optionally,
   conditioning the extracted vitreous humor by subjecting the said extracted vitreous humor to an atmosphere of a predetermined CO₂ concentration for a predetermined period of time.
14. Use of a sample fluid according to item 12, or a sample fluid prepared by the method according to item 13 in the method according to item 1.
15. Use of a buffer fluid in the method according to item 1 to 11, wherein the buffer fluid comprises sodium, potassium, calcium, magnesium, chloride, bicarbonate, phosphate, lactate, glucose and optionally a preservative such as azide.
16. Use of an apparatus (10) in the method according to items 1 to 11, wherein the apparatus is configured for stimulating the behavior of a substance in an eye and comprises:
   at least one sample cell (26) having a sample chamber (28) which is at least partly delimited by a semi-permeable membrane (30), wherein the sample cell is configured to receive a sample comprising a substance and a sample fluid constituting a simulated physiological environment of an eye; and
   a buffer supply system (16) configured to guide a flow (34) of buffer fluid over the outer surface of the semi-permeable membrane (30) for a predetermined period of time (Tⱼ).
17. The method of item 5, wherein the buffer solution comprises
   between about 50 mmol/L and about 250 mmol/l sodium,
   between about 1.0 mmol/L and about 25 mmol/L potassium,
   between about 0.2 mmol/L and about 5.0 mmol/L calcium,
   between about 0.1 mmol/L and about 4.0 mmol/L magnesium,
   between about 25 mmol/L and about 200 mmol/L chloride,
   between about 5 mmol/L and about 100 mmol/L bicarbonate,
   between about 0.1 mmol/L and about 10 mmol/L phosphate,
   between about 0.8 mmol/L and about 20 mmol/L lactate,
   between about 0.8 mmol/L and about 20 mmol/L glucose,
   between about 0.1 mmol/L and about 10 mmol/L glutathione disulfide,
and optionally a preservative, e.g., between about 0.5 mmol/L and about 10 mmol/L azide.

1. Method for simulating and analyzing the behavior of a substance in an eye, comprising the steps of:
a) providing at least one sample cell having a sample chamber which is at least partly delimited by a semi-permeable membrane;
b) filling the sample chamber with a sample comprising at least one substance and a sample fluid constituting a simulated physiological environment of an eye;
c) guiding a flow of buffer fluid over the outer surface of said semi-permeable membrane for a predetermined period of time (Tj); and
d) analyzing the sample.

2. The method according to claim 1, wherein, before performing step c), the buffer fluid is conditioned by subjecting the buffer fluid to an atmosphere of a predetermined CO2 concentration for a predetermined period of time.

3. The method according to claim 1, wherein said flow of buffer fluid in step c) is a laminar flow and/or wherein the buffer fluid flow has a flow rate in the range between 6 mL/min to 10 mL/min.

4. The method according to claim 1, wherein the substance is an active agent and wherein the sample fluid is or simulates vitreous humor.

5. The method according to claim 1, further comprising a step of providing the sample fluid, including the sub-steps of:
extracting vitreous humor from at least one isolated vertebrate eye;
centrifuging the extracted vitreous humor to separate a clear fraction of the extracted vitreous humor;
filtering the extracted vitreous humor; and
optionally mixing the extracted vitreous humor with a buffer solution to provide the sample fluid.

6. The method according to claim 1, wherein the sample chamber has a volume in the range of 4 mL to 6 mL and/or wherein the semi-permeable membrane has a molecular weight cut off in the range of 50 kDa to 100 kDa.

7. The method according to claim 1, further comprising a step of labelling the substance with a fluorescent dye.

8. The method according to claim 1, wherein the buffer fluid, upon being guided over the semi-permeable membrane of the sample cell:
sets a predetermined pH condition of the sample received in the sample chamber; and
receives substances which diffuse out of the sample chamber over the semi-permeable membrane into the buffer fluid.

9. The method according to claim 1, wherein the buffer fluid comprises at least one cationic species selected from the group of sodium, potassium, calcium, magnesium, and at least one anionic species selected from the group of chloride, bicarbonate, phosphate, lactate, glucose and optionally a preservative.

10. The method according to claim 1, wherein the step of analyzing the sample comprises determining the physicochemical stability of the at least one substance.

11. The method according to claim 1, wherein more than one sample cell is provided in step a), and wherein the predetermined period of time (Tj) in step c) of guiding a flow of buffer fluid over the outer surface of said semi-permeable membrane of each sample cell is different for the different sample cells.

12. Sample fluid prepared by extracting vitreous humor from vertebrate eyes; centrifuging the extracted vitreous humor to separate a clear fraction of the extracted vitreous humor; and filtering the extracted vitreous humor, and optionally, conditioning the extracted vitreous humor by subjecting the said extracted vitreous humor to an atmosphere of a predetermined CO₂ concentration for a predetermined period of time.

13. The method according to claim 1, wherein the sample fluid is provided by a method, comprising the steps of:
extracting vitreous humor from vertebrate eyes;
centrifuging the extracted vitreous humor to separate a clear fraction of the extracted vitreous humor; and
filtering the extracted vitreous humor, and optionally,
conditioning the extracted vitreous humor by subjecting the said extracted vitreous humor to an atmosphere of a predetermined CO₂ concentration for a predetermined period of time.

14. (canceled)

15. The method according to claim 1, wherein the buffer fluid comprises sodium, potassium, calcium, magnesium, chloride, bicarbonate, phosphate, lactate, glucose and optionally a preservative.

16. The method according to claim 1, which comprises use of an apparatus configured for stimulating the behavior of a substance in an eye and comprises:
at least one sample cell having a sample chamber which is at least partly delimited by a semi-permeable membrane, wherein the sample cell is configured to receive a sample comprising a substance and a sample fluid constituting a simulated physiological environment of an eye; and
a buffer supply system configured to guide a flow of buffer fluid over the outer surface of the semi-permeable membrane for a predetermined period of time (Tj).

17. The method of claim 5, wherein the buffer solution comprises
between about 50 mmol/L and about 250 mmol/l sodium,
between about 1.0 mmol/L and about 25 mmol/L potassium,
between about 0.2 mmol/L and about 5.0 mmol/L calcium,
between about 0.1 mmol/L and about 4.0 mmol/L magnesium,
between about 25 mmol/L and about 200 mmol/L chloride,
between about 5 mmol/L and about 100 mmol/L bicarbonate,
between about 0.1 mmol/L and about 10 mmol/L phosphate,
between about 0.8 mmol/L and about 20 mmol/L lactate,
between about 0.8 mmol/L and about 20 mmol/L glucose,
between about 0.1 mmol/L and about 10 mmol/L glutathione disulfide,
and optionally a preservative.

18. The method according to claim 4, wherein the substance is an active agent for topical, systemic, intravitreal, intrathecal, subcutaneous, subconjunctival, retrobulbar, or intracameral administration.

19. The method according to claim 8, wherein the substances which diffuse out of the sample chamber are precipitation or degradation products from the sample received in the sample chamber.

20. The method according to claim 9, wherein the preservative is an azide.
